SEQUENCE LISTING (8 sequences)

SEQ ID NO: 1 - protein, 128 aa, Homo sapiens
  PEPTIDE (1)..(128)
MIFLKLIKSIVIGLGLVSAIQAAPASSIGSSASASSSSESSQATIPNDVTLGVKQIPNIFNDSAVDANAAAKHPLEKRFVNQHLCGSHLVEALYLVCGERGFFYTPKGIVEQCCTSICSLYQLENYCN

SEQ ID NO: 2 - protein, 128 aa, Homo sapiens
  PEPTIDE (1)..(128)
MIFLKLIKSIVIGLGLVSAIQAAPASSIGSSASASSSSESSQATIPNDVTLGVKQIPNIFNDSAVDANAAAKHPLENRFVNQHLCGSHLVEALYLVCGERGFFYTPKGIVEQCCTSICSLYQLENYCN

SEQ ID NO: 3 - protein, 116 aa, Homo sapiens
  PEPTIDE (1)..(116)
MTSKTIPAMLAIITVAYLCALPHAHARSTQGYGRMDRILAALKTSPMEPSAALAVENGTTHPLGKRFVNQHLCGSHLVEALYLVCGERGFFYTPKGIVEQCCTSICSLYQLENYCN

SEQ ID NO: 4 - protein, 116 aa, Homo sapiens
  PEPTIDE (1)..(116)
MTSKTIPAMLAIITVAYLCALPHAHARSTQGYGRMDRILAALKTSPMEPSAALAVENGTTHPLGNRFVNQHLCGSHLVEALYLVCGERGFFYTPKGIVEQCCTSICSLYQLENYCN

SEQ ID NO: 5 - DNA, 384 bp, Homo sapiens
atgatctttctgaagttgatcaagtctatcgtgatcggtctgggtctggtttctgccatccaggccgctccagcctcttctatcggttcttctgcctctgcctcttcttcttctgagtcttctcaggccaccattccaaacgacgttaccctgggtgttaagcagatcccaaacatcttcaacgactctgccgttgacgccaacgctgctgctaagcacccactggagaagagattcgtgaaccagcacctgtgtggttctcacctggttgaggccctgtacctggtttgcggtgagagaggattcttctacaccccaaagggtatcgttgagcagtgctgcacctctatctgttctctgtaccagctggagaactactgcaac

SEQ ID NO: 6 - DNA, 384 bp, Homo sapiens
atgatctttctgaagttgatcaagtctatcgtgatcggtctgggtctggtttctgccatccaggccgctccagcctcttctatcggttcttctgcctctgcctcttcttcttctgagtcttctcaggccaccattccaaacgacgttaccctgggtgttaagcagatcccaaacatcttcaacgactctgccgttgacgccaacgctgctgctaagcacccactggagaacagattcgtgaaccagcacctgtgtggttctcacctggttgaggccctgtacctggtttgcggtgagagaggattcttctacaccccaaagggtatcgttgagcagtgctgcacctctatctgttctctgtaccagctggagaactactgcaac

SEQ ID NO: 7 - DNA, 348 bp, Homo sapiens
atgacctcgaagaccatcccagccatgctggccatcattaccgttgcctacctgtgtgctctgccacacgcccacgctagatctacccagggttacggtagaatggacagaatcctggccgccctgaagacctctccaatggagccatctgccgccctggccgttgagaacggaaccacccacccactgggtaagagattcgtgaaccagcacctgtgtggttctcacctggttgaggccctgtacctggtttgcggtgagagaggattcttctacaccccaaagggtatcgttgagcagtgctgcacctctatctgttctctgtaccagctggagaactactgcaac

SEQ ID NO: 8 - DNA, 348 bp, Homo sapiens
atgacctcgaagaccatcccagccatgctggccatcattaccgttgcctacctgtgtgctctgccacacgcccacgctagatctacccagggttacggtagaatggacagaatcctggccgccctgaagacctctccaatggagccatctgccgccctggccgttgagaacggaaccacccacccactgggtaacagattcgtgaaccagcacctgtgtggttctcacctggttgaggccctgtacctggtttgcggtgagagaggattcttctacaccccaaagggtatcgttgagcagtgctgcacctctatctgttctctgtaccagctggagaactactgcaac

(19) United States
(12) Patent Application Publication
(10) Pub. No.: US 2006/0234351 A1
(43) Pub. Date: Oct. 19, 2006

(54) Yeast protein expression secretion system

(76) Inventors: Maharaj K. Sahib, Auranagabad (IN); Edupuganti B. Raju, Aurangabad (IN)

Correspondence Address: O M (Sam) Zaghmout;Bio Intellectual Property Services (Bioips), 8509 Kernon Ct, Lorton, VA, 22079, US

(21) Appl. No.: 10/524,036

(22) PCT Filed: Sep. 8, 2003
(86) PCT No.: PCT/IB03/03773

§ 371 Date: Feb. 9, 2005

Related U.S. Application Data
(60) Provisional application No. 60/410,774, filed on Sep. 13, 2002.

(51) Int. Cl. C12P 21/06; C12N 1/18; C07H 21/04; C12N 15/74; A61K 38/28; C07K 14/62

(52) U.S. Cl. 435/69.4; 530/303; 435/483; 435/254.2; 435/254.21; 435/254.23; 536/23.5

(57) ABSTRACT

This invention discloses novel prepro-insulin polypeptides. The polypeptides consist of an N-terminal region, derived from N-terminal regions of secretory proteins, and a downstream insulin polypeptide region. The N-terminal region directs the polypeptides efficiently into the secretory pathway of yeasts. Modifications at the N-terminal region, just adjacent to the insulin polypeptide region, further increase the efficiency of secretion and improves the final yield of secreted insulin. The patent also discloses expression systems for the expression of said polypeptides under the regulation of yeast derived alcohol inducible promoters. Thus a combination of such promoters and precursors with the said N-terminal regions appear to function as very high yielding expression systems in yeasts.

### FIELD OF INVENTION

The present invention relates to novel expression systems for high level and efficient expression of insulin as prepro-polypeptides in yeast. These pre-propolypeptides are efficiently secreted into the extracellular medium, from where they may conveniently isolated, converted to native insulin and purified further.

### BACKGROUND TO THE INVENTION

Insulin is a protein harmone that is secreted by the beta cells of the pancreas and plays a key role in the homeostasis of blood sugar. A key etiology of diabetes is the reduced or the complete cessation of insulin production and secretion by the beta cells, as well as resistance to its effects in the peripheral tissues. Thus treatment with insulin remains the most effective therapeutic strategy for diabetes, to ameliorate its symptoms as well as its associated complications. The early treatments with insulin involved the use of the harmone isolated from bovine or porcine sources or from the pancreas of human cadavers. The preparation of such insulins, from human, bovine or porcine sources, is a highly cumbersome process, associated with difficult purification procedures, very low yields, and large amounts of impurities. Also, insulins from non-human sources may cause potentially allergic reactions. However, the tools of recombinant DNA technology address most of these difficulties by providing the means to obtain human insulin conveniently, in very high yields and with very high degree of purity.

The methods of recombinant DNA technology generally consist of isolating or synthesizing the gene that encodes a particular protein of interest and cloning the same into a suitable “heterologous host”. The host is then cultured under suitable conditions to express the protein to very high levels. The protein may then be conveniently isolated and purified from the culture medium. But several factors effect the final yield and purity of even recombinantly expressed protein. These factors basically depend on the choice of the expression system, particularly the host culture, employed for the expression of the protein. The various strains of the bacterium E. coli by far remain “the hosts of choice” for the heterologous expression of proteins. The reasons for this is the rapid generation time of E. coli and the consequent easy availability of a large biomass, the ease of genetic manipulation for generating a high expressing strain, the availability of a plethora of “expression vectors” tailored to the needs of specific E. coli strains for optimal expression etc. Yet E. coli expression systems are not without their disadvantages, the most important being the absence of “modification” systems that would otherwise chemically modify proteins of plant and animal origin and that may be crucial to protein function. In addition, quite often proteins are expressed as inactive aggregates (“inclusion bodies”) inside the E. coli. Isolation of active protein from such inclusion bodies involves an additional step in the purification procedures, which in turn effects the final yield of the protein, as well the overall cost of isolation. These particular disadvantages may be overcome by expressing a protein in “higher” cellular hosts—either animal or plant cell culture systems. But the latter expression hosts are highly expensive, as well as yield much lower biomass as compared to E. coli strains. Yeast strains combine the advantages of the above distinct host systems. On the one hand they more closely mimic the native physiology of a plant/animal protein then does E. coli, on the other hand their ease of handling, ease of cultivation, much faster growth and much greater economy are typical of the advantages provided by E. coli.

Several factors though, effect the expression of proteins in yeast as well. These factors include, but are not confined to:
1) The choice of the gene regulatory sequences, such as promoters, that control the expression of an heterologous protein. The promoter sequences employed for controlling heterologous expression must typically be “strong,” in that they effect very high expression of the protein, and suitably “controllable”, whereby the expression may at first be efficiently repressed until an optimum biomass of the culture is reached and then quickly “switched on” to effect protein expression.
2) Efficient secretion of the expressed heterologous protein. Secretion of the expressed protein (“extracellular” expression) is often preferred over intracellular expression as the latter would first entail breaking open the cell, thus disgorging the entire cellular contents, and then isolating the desired protein from the cesspool of cellular material and debris. Yet efficient secretion of a protein in turn depends on several factors including: a) the choice of the signal sequences—peptide sequences which are usually the N-terminal regions of naturally secreted proteins, and which direct the protein into the cellular secretory pathway and, b) the specific components of the secretory pathway that interact with signal sequences and effect the secretion of the attached protein.

Clearly there exists an enormous scope for the development of expression systems for improved large-scale production of proteins. The present invention provides such a system for the expression of insulin in yeast.

The U.S. patent H245 discloses a plasmid capable of replication and expression in E. coli of a human preproinsulin polypeptide, while U.S. Pat. No. 4,431,740 describes a transfer vector carrying a cDNA of human pre-proinsulin and proinsulin. U.S. Pat. No. 4,916,212 claims a DNA sequence encoding an insulin precursor of the formula B(1-29)-(Xn-Y)ₘ-A(1-21) where m can be 0 or 1, n=0 to 33 and X and Y represent amino acid sequences specifically defined in the patent, while U.S. Pat. Nos. 5,202,415 and 5,324,641 describe, respectively, insulin precursors and DNA sequences of B(1-29)-X1-X2-Y1-Y2-B(1-21), where Y1 and Y2 each represent basic amino acid residues. U.S. Pat. No. 5,962,267 claims a precursor of the formula B-Z-A where B and A chains are respectively human insulin chains and Z is a specifically defined peptide. U.S. Pat. Nos. 4,914,026 and 5,015,575 teach the expression and secretion of human insulin chains in yeast, particularly Saccharomyces, under the control of a promoter functional in yeast and the secretion being directed by a yeast alpha-factor leader sequence fused to the insulin precursor. Also U.S. Pat. No. 6,337,194 describes the expression in yeast of a polypeptide of the general formula B-Z-A where B and A chains are insulin chains and Z is a peptide region with sequences that contain at least one proteolytic cleavage site. Z may further comprise an affinity polypeptide tag for the isolation and purification of the secreted product. The U.S. Pat. Nos. 5,389,525, 5,240,838 and 5,741,672 describe the use of formaldehyde dehydrogenase and methanol oxidase respectively in the expression of proteins in the yeast strain Hansenula polymorpha. On the other hand U.S. Pat. Nos. 5,414,585, 5,395,922 and 5,510,249 describe a polypeptide, consisting of signal and leader peptide sequences and a heterlogous polypeptide, that is efficiently processed prior to the secretion of the heterologous protein in yeast. Furthermore the U.S. Pat. Nos. 5,672,487 and 5,741,674 describe a process for the recombinant production of protein in yeast, whereby the yeast strain is transformed with an expression cassette consisting of a leader, adapter and a processing signal preceding the heterelogous polypeptide. The patent specifically describes the use of an adapter polypeptide having an alpha-helical structure.

The present invention describes the expression of insulin, particularly human insulin, B and A chains as a fusion protein, fused to signal peptide sequences, under the control of alcohol inducible promoters, such that the fusion polypeptide is very efficiently expressed and secreted from yeasts.

### SUMMERY OF THE INVENTION

The present invention describes processes for the expression in yeast, of insulin as a prepro-polypeptide, said polypeptide consisting of a signal sequence, derived from the Schwanniomyces occidentalis glucoamylase or Carcinus maenas crustacean hyperglycemic harmone signal-leader sequence, and present at N-terminus of an insulin polypeptide of the formula:
B(1-29)-A(1-21)
where B(1-29) and A(1-21) refer to the human insulin B chain from amino acid 1 to amino acid 29 and the human insulin A chain from amino acid 1 to amino acid 21 respectively.

The said process consists of cloning a gene encoding said prepro-polypeptide into a yeast expression system under the control of a yeast alcohol inducible promoter, culturing the yeast in an appropriate culture medium, isolating the said polypeptide from the culture medium, and processing the same to get rid of the signal peptide region and obtain the final native form of the human insulin protein.

### DETAILED DESCRIPTION OF THE INVENTION

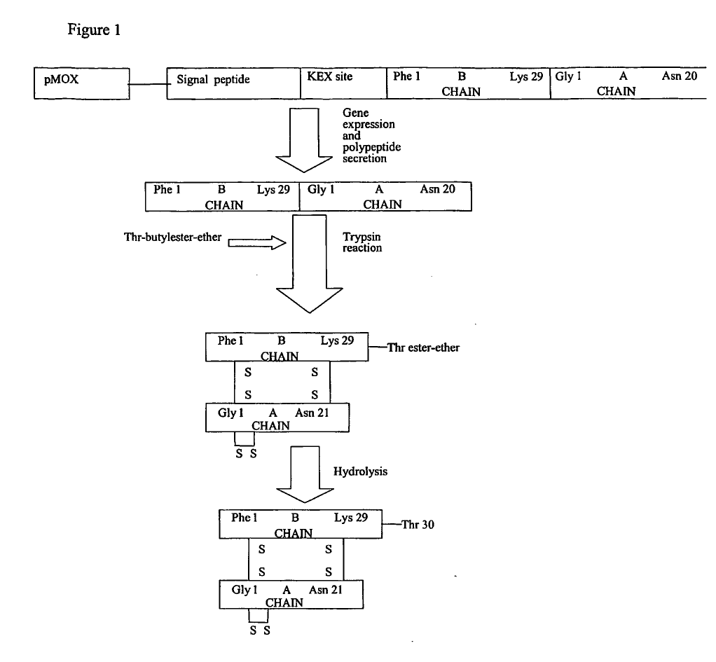

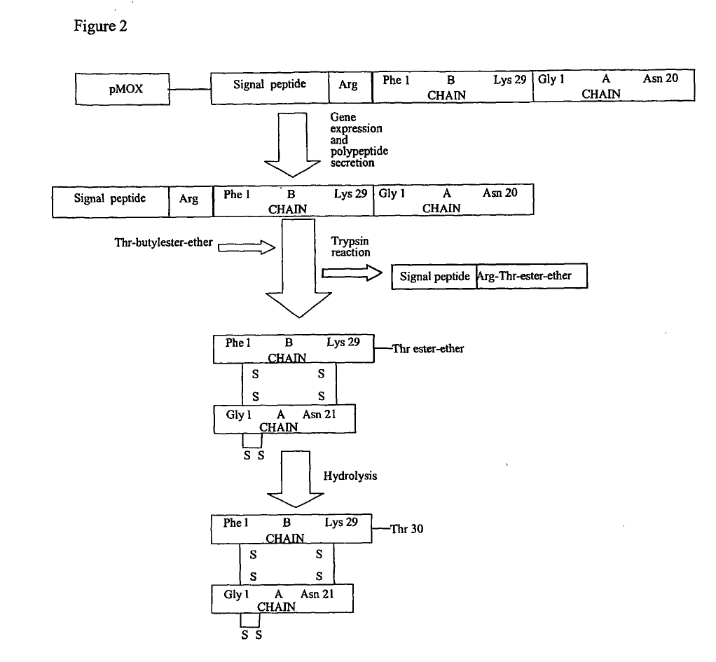

The present invention provides a composite system for the expression and secretion of insulin, particularly human insulin, in yeast. It consists of expressing insulin as a “prepro”-polypeptide, consisting of two distinct entities—an “insulin region” (the “pro” region) and a “signal peptide region” (the “pre” region). The pro-polypeptide region has the formula: B(1-29)-A(1-21), where B(1-29) is the B chain polypeptide of insulin, preferably human insulin, from amino acid 1 to amino acid 29 and A(1-21) is the A chain polypeptide of insulin, preferably human insulin, from amino acid 1 to amino acid 21. The amino acid 29 of the B chain is connected directly to the amino acid 1 of the A chain by means of a peptide bond. The said pro-polypeptide B(1-29)-A(1-21) may be converted into the “native” insulin—B(1-30):::A(1-21) (where the B and the A chain are no longer connected by a peptide bond and instead have 2 interchain and 1 intrachain disulfide bonds) by means of a “transpeptidation” reaction with Threonine-butylester-butylether, in the presence of the proteolytic enzyme trypsin, followed by hydrolysis (Refer U.S. Pat. No. 4,343,898 or 4,489,159). The second entity of the prepro-polypeptide—the signal peptide region—is the region that directs the polypeptide into the yeast secretory pathway. This region is N-terminus to the insulin polypeptide region and connected to the amino acid 1 of the B chain by means of a peptide bond. The signal peptide may be derived either from Schwanniomyces occidentalis glucoamylase signal peptide sequence or Carcinus maenas crustacean hyperglycemic harmone signal peptide sequence. In one embodiment of the present invention the signal peptide region carries the Kex protease site, that could interact with the Kex protease present in the secretory pathway of the yeast expression host. Such an interaction would result in the cleavage of the signal peptide region during the secretion of the heterologous polypeptide. Hence, in this case the polypeptide is secreted into the culture medium only as the pro-polypeptide viz. B(1-29)-A(1-21). This may then be isolated and converted to the native form (B(1-30):::A(1-21)) by the said transpeptidation and hydrolysis reactions (depicted in FIG. 1). In a second embodiment of the present invention, the signal peptide region does not contain the kex protease site. In this case the polypeptide secreted into the culture medium is the prepro-polypeptide viz. SP-B(1-29)-A(1-21), where SP is the signal peptide region that remains attached to the amino acid 1 of the B chain by means of the peptide bond. This second embodiment would hence require the in vitro removel of the signal peptide region as well as conversion of the B(1-29)-A(1-21) into the “native” form—B(1-30):::A(1-21). Hence in a further aspect of the second embodiment, the prepro form carries either one basic amino acid residue (arginine or lysine) or methionine immediately adjecent and N-terminus to the B(1-29)-A(1-21) region. The said basic amino acid residue or methionine residue aid the removel the signal peptide region from the B(1-29)-A(1-21) region by means of a chemical reaction with either trypsin or cyanogen bromide respectively. Of the two general embodiment described above, the second embodiment is preferred over the first because, while the the second embodiment does require the additional reaction to remove the signal peptide region, we observe that the yields of the polypeptide obtained by following the first embodiment are much lower then those obtained from the second embodiment. This may, in part, be due to the increased intracellular retention of the heterologous protein in the first embodiment. This increased retention may be a result of the increased interactions with the Kex protease in the secretory pathway, and a consequent reduced levels of protein secreted into the culture medium. On the other hand, since the heterologous polypeptides (the prepro-polypeptides) of the second embodiment do not carry the Kex protease site, there may be reduced interactions between the polypeptide and the intracellular protease, and a consequent increased levels of secreted polypeptide. Furthermore, in the case of the second embodiment, between the use of either the basic amino acid residue or methionine, we prefer the use of the basic amino acid (cleavable with trypsin), because then the secreted form viz.—SP-B(1-29)-A(1-21) may be converted directly into the “native” form B(1-30):::A(1-21) by the same transpeptidation reaction required for the conversion of B(1-29)-A(1-21) to the native form—B(1-30):::A(1-21). Thus a single trypsin-transpeptidation reaction would remove the signal peptide (SP) region, as well as convert the pro form [(B(1-29)-A(1-21)] into the native form [(B(1-30):::A(1-21)] (as depicted in FIG. 2). Seq ID 1 and 3 are examples of the polypeptides representing the first embodiment (viz. with Kex site) and Seq ID 2 and 4 are examples of the polypeptides representing the second embodiment (viz. without kex site). In seq ID 1 and 2 the signal peptide region is derived from Schwanniomyces occidentalis glucoamylase signal peptide sequence and in Seq ID 3 and 4 the signal peptide region is derived from Carcinus maenas crustacean hyperglycemic harmone signal peptide sequence. Seq ID 5, 6, 7, 8 are examples of DNA sequences encoding the polypeptides represented in Seq ID 1, 2, 3, 4 respectively.

The DNA sequences encoding the prepro-polypeptides described above were cloned into a yeast expression vector under the control of alcohol inducible promoters. Examples of such promoters include the promoters native to the yeast methanol oxidase (MOX), formaldehyde dehydrogenase (FMDH), formate dehydrogenase (FMD) and dihydroxyacetone synthetase (DHAS) genes. The recombinant expression vectors, carrying the DNA sequences of the prepro-polypeptides under the control of the alcohol inducible promoters, were then transformed into appropriate yeast host strains. Examples of such host strains include genera of Hansenula, Saccharomyces, Pichia, Kluyveromyces. The transformed yeast were then cultured in an appropriate culture medium, the polypeptides were isolated from the medium and then converted into the native form.

The present invention thus provides a composite expression system for the very high expression of human insulin. The expression system consists of an alcohol inducible promoter and the DNA sequence of a “prepro”-polypeptide. The prepro-polypeptide in turn consists of the DNA sequence encoding the insulin polypeptide region [B(1-29)-A(1-21)] and the DNA sequence encoding either the Schwanniomyces occidentalis glucoamylase signal peptide sequence or the Carcinus maenas crustacean hyperglycemic harmone signal peptide sequence. The prepro-polypeptide may or may not carry the sequence recognized by the Kex protease site between the signal peptide region and the insulin polypeptide region. If the Kex protease site is absent, then either one basic amino acid residue (lysine or arginine) or one methionine residue is present between the signal peptide region and the insulin polypeptide region. In either case the expressed polypeptide is secreted into the intracellular medium, conveniently isolated and further processed to obtain the native insulin. The processing mechanisms are depicted in FIGS. 1 and 2.

The examples that follow, figures and Seq IDs merely illustrate the invention in greater detail, but in no way restrict the scope of the same.

### EXAMPLE 1

Construction of the Recombinant Vector Carrying the Prepro-Polypeptides.

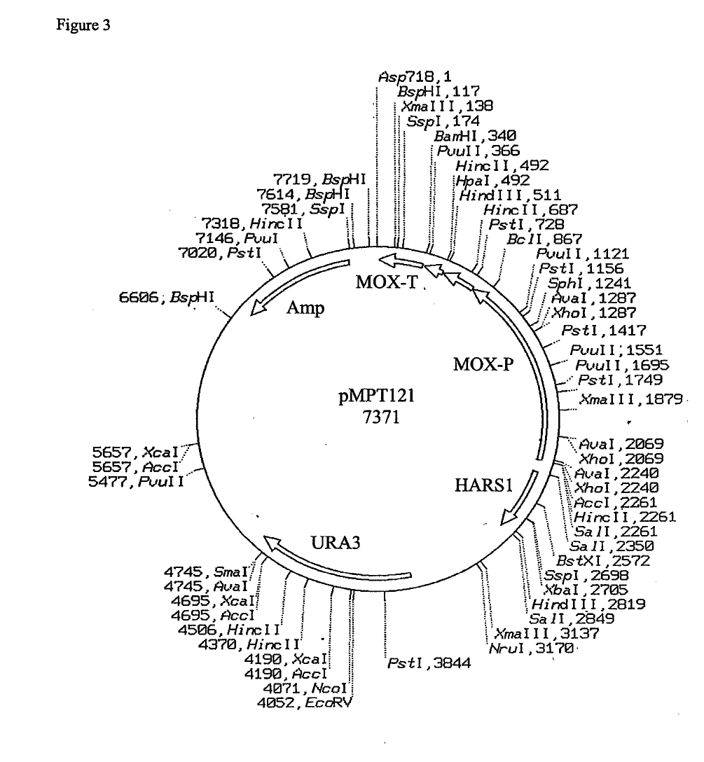

Seq ID 1, 2, 3 and 4 correspond to the amino acid sequences of the prepro-polypeptides InGa, InGa−, InCh, InCh−. In the case of Seq ID 1 and 2, the peptide region from amino acid 1 to 78 is the signal peptide region that ensures the secretion of the heterologous proteins. On the other hand the peptide region 79-107 of Seq ID 1 and 2 corresponds to amino acids 1-29 of the human insulin B chain, while the peptide region 108 to 128 of Seq ID 1 and 2 corresponds to amino acids 1-21 of the human insulin A chain. Similarly, in the case of Seq ID 3 and 4, the peptide region from amino acids 1-66 corresponds to the signal peptide regions, whereas the peptide region 67-116 corresponds to the insulin B and A chain regions as above. The signal peptide regions of Seq ID 1 and 2 are derived from Schwanniomyces occidentalis glucoamylase signal peptide sequence, with Seq ID 1 possessing the kex site, whereas Seq ID 2 not possessing the same. On the other hand, the signal peptide regions of Seq ID 3 and 4 are derived from the Carcinus maenas crustacean hyperglycemic harmone signal sequence, with Seq ID 3 possessing the kex site, whereas Seq ID 4 not possessing the same. The Seq ID 5, 6, 7, 8 correspond to the oligonucleotides that encode said prepro-polypeptides InGa, InGa−, InCh, InCh−(defined by Seq ID 1, 2, 3, 4). These oligonucleotides were chemically synthesized and designed to have those codons that are most optimally expressed in the yeast Hansenula polymorpha. The oligonucleotides were cloned into the EcoRI and BamH1 restriction enzyme sites of the plasmid expression vector pMPT121 (FIG. 3) by carrying out restriction enzyme digestion and ligation reactions by methods well known to those of ordinary skill in the art (“Molecular Cloning: A Laboratory Manual” by J. Sambrook, E. F. Fritsch and T. Maniatis, II edition, Cold Spring Harbour Laboratory Press, 1989). The pMPT121 plasmid expression vector is based on a pBR322 plasmid and contains the following elements:
standard E. coli pBR322 skeleton including E. coli origin of replication (ori).
ampicillin resistance gene for selection of transformed E. coli.
auxotrophic selective marker gene complementing the auxotrophic deficiency of the host—Hansenula polymorpha, (H. polymorpha) (URA3 gene).
H. polymorpha Autonomously Replicating Sequence (HARS).
an expression cassette containing the MOX promoter and the MOX terminator for insertion of the gene construct and controlling the expression of the cloned heterlogous polypeptides in the said yeast strain.

The individual ligation reactions were then transformed into E. coli hosts by methods well known to those skilled in the art (“Molecular Cloning: A Laboratory Manual” by J. Sambrook, E. F. Fritsch and T. Maniatis, II edition, Cold Spring Harbour Laboratory Press, 1989). Various E. coli clones carrying the recombinant plasmids were cultured and the plasmids isolated by methods well known in the art (“Molecular Cloning: A Laboratory Manual” by J. Sambrook, E. F. Fritsch and T. Maniatis, II edition, Cold Spring Harbour Laboratory Press, 1989). The isolated recombinant plasmids were then confirmed to be carrying the above oligonucleotides, encoding the respective prepro-polypeptides, by DNA sequencing.

### EXAMPLE 2

Transformation of a Yeast Strain with the Recombinant Vectors Carrying the Insulin Precursor Sequences.

The recombinant expression plasmids each carrying the oligonucleotides encoding the prepro-polypeptides InGa, InGa−, InCh, InCh−, were then transformed into the yeast strain H. polymorpha that is an ura3 auxotrophic mutant deficient in orotidine-5′-phosphate decarboxylase by methods known in the art (Hansenula polymorpha: Biology and Applications, Ed. G. Gellissen. Wiley-VCH, 2002). The resulting recombinant clones were then further used for the expression of the said polypeptides.

### EXAMPLE 3

Expression of the Insulin Precursors in Yeast.

The yeast transformants thus obtained were then used for the expression of the insulin prepro-polypeptides InGa, InGa−, InCh, InCh−. The expression conditions were:
a) Preculture: Single clones, each carrying the expression vector carrying the oligonucleotide sequences encoding the prepro-polypeptides InGa, InGa−, InCh, InCh−, were inoculated into 100 ml of autoclaved 2× YNB/1.5% glycerol medium in a 500 ml shake flask with baffles. The composition of the 2× YNB/1.5% is 0.28 g yeast nitrogen base, 1.0 g ammonium sulfate, 1.5 g glycerol and 100 ml water. The cultures were incubated for about 24 h at 37° C. with 140 rpm shaking until an O.D₆₀₀ of 3-5 is reached. The final pH after incubation is around 2.9-3.
b) Culture: 2×450 ml of autoclaved SYN6/1.5% glycerol media in 2×2000 shake flasks with baffles were inoculated with 20-50 ml of each of the above preculture. The cultures were then incubated for 48 h at 30° C. and 140 rpm. The composition of the SYN6/1.5% glycerol medium is NH₄H₂PO₄—13.3. g, MgSO₄×7H₂O—3.0 g, KCl—3.3 g, NaCl—0.3 g, glycerol—15.0 g, water 1000 liters. In addition the following solutions (filter sterilized) were added to the autoclaved media: CaCl₂ solution—6.7 ml, microelement solution—6.7 ml, vitamin solution—6.7 ml, trace element solution—3.3 ml.

### EXAMPLE 4

Isolation and Estimation of Insulin Polypeptide Precursors.

1.5 ml of the supernatants from the cultures expressing the secreted prepro-polypeptides, InGa, InGa−, InCh, InCh− were isolated by centrifugation and quantified on an analytical RP-HPLC column (Nucleosil C18, 5 μm, 2 mm×50 mm). The buffers employed for analysis were: Buffer A: 10% Acetonitrile, 0.1% trifluoroacetic acid in water and Buffer B: 80% acetonitrile, 0.1% trifluoroacetic acid in water. The yields of each are expressed in Table 1, as total insulin components normalized with dry cell weight, and expressed as % yield, with the yields of precursors having the processing site (InGa, InCh) taken as 100%.

TABLE 1

| Prepro-polypeptides | % Yield |
|---|---|
| InGa | 100 |
| InGa− | 135 |
| InCh | 100 |
| InCh− | 127 |

Thus the yield of the InGa− and InCh− (which do not have the Kex site) are, respectively, 35% and 27% higher then those of InGa and InCh.

### EXAMPLE 5

Isolation, Purification and Conversion of the Prepro-Polypeptides to “Native” Insulin.

Cell Clarification.

Culture supernatants from example 3 were pooled and clarified by centrifugation. The prepro-polypeptides were then isolated from this diluted supernatant by Cation exchange chromatography.

Cation Exchange Chromatography.

A Chromatography column of 26 mm×50 mm dimensions was packed with 25 ml cation exchange SP-Sepharose fast flow (Pharmacia) resin and equilibrated with 20 mM citrate buffer at pH 4.0. The diluted supernatents were applied to the cation exchange column at pH 4.0 and a flow rate of 200 cm/h. The columns were then washed with 20 mM citrate buffer (5 Column Volumes) at 200 cm/h. The bound prepro-polypeptides were eluated with a buffer containing 100 mM tris HCl at pH 7.5, at a flow rate of 100 cm/h. About 306 mg of prepro-polypeptides were obtained when about 348 mg of prepro-polypeptides was applied to the column.

Isoelectric Precipitation.

300 mg of zinc chloride was added to 306 mg of prepro-polypeptides obtained from the above described cation exchange chromatography. The pH was adjusted to 6.0 with HCl to precipitate the prepro-polypeptides from the pool. The reactions were kept at 8° C. for 12 hours followed by centrifugation and then drying.

Transpeptidation.

About 300 mg of precipitated prepro-polypeptides from above were then dissolved and incubated at 12° C., in a reaction mixture containing 2.36 ml of Dimethyl sulfoxide/Methanol (50/50 v/v), 1.5 g of L-Threonine-t-butylester-t-butyl ether, 1.44 ml milliQ water and 30 ul of acetic acid. The reactions were chilled for 5 min in ice. 15 mg of trypsin (from bovine pancreas dissolved in 0.255 ml of 50 mM Calcium acetate and 0.05% acetic acid) was added, pH adjusted to 7.3 and the reaction mixture was incubated at 12° C. for about 3 hours. The reactions were quenched by reducing the pH to 3.0 with 1N HCl. This reaction results in the conversion of the prepro-polypeptides to insulin-t-butylester-t-butyl ether (refer FIGS. 1 and 2).

Purification of Insulin -t-butylester-t-butyl ether.

From the above reaction mixtures, about 234 mg of the t-butyl ester-t-butyl ether derivatives were diluted 10 fold with 10% 2-propanol containing 0.01% TFA and then applied to a chromatography column of 20 mm×50 mm dimensions and packed with 25 ml reverse phase Amberchrome CG-300 SD resin. The column had been pre-equilibrated with buffer A (composition below) and the reaction mixtures applied at a flow rate of 100 cm/h. The column is equipped with a binary gradient solvent delivery system and an online ultraviolet detector. The buffers used were, Buffer A: 10% v/v 2-propanol, 0.1% trifluoro acetic acid (TFA) and Buffer B: 80% v/v 2-propanol, 0.1% trifluoroacetic acid (TFA). After loading, the column was washed with 5 Column Volumes of 20% buffer B at a flow rate of 100 cm/h. The insulin-t-butyl ester-t-butyl ether derivatives were eluted with a linear gradient of 20% to 50% buffer B in 7.5 column volumes at a flow rate of 100 cm/h. The fractions containing pure insulin-ester-ethers were pooled, 2-propanol was removed under reduced pressure and the aqueous phase lyophilized to obtain dry insulin-t-butyl ester-t-butyl ether.

Hydrolysis.

About 180 mg of lyophilized insulin-t-butyl-ester-t-butyl-ether was hydrolyzed to “native” insulin in a 100 ml round bottom flask by dissolving it in anhydrous trifluoroacetic acid at a concentration of 10 mg insulin derivative per ml TFA, in presence of 0.5 mg tryptophan per ml of TFA. The reaction mixtures were kept at 25° C. for 20 min. TFA was removed from the reaction mixture under reduced pressure in a Buchi rota evaporator and resuspended the residue mass in 20 ml 1% acetic acid (v/v).

Final HPLC Purification.

About 170 mg insulin obtained from the hydrolysis reaction (described above) was filtered to remove particulate matter and applied to a C18, 10 mm×250 mm, Vydac reverse phase HPLC column equipped with a binary gradient pump and an online ultraviolet detector at 280 nm. The buffers used were: Buffer A containing 0.2M sodium sulfate, 20% Acetonitrile and 0.01% TFA, and Buffer B—mixture of 50% Acetonitrile, 50% water and 0.01% TFA. After loading, the column was washed with 1 Column Volume of 20% buffer B at a flow rate of 4 ml/min. Insulin was isolated achieved with gradient elution that followed washing. During elution the concentration of buffer B increased from 20% to 40% over a period of 300 min at a flow rate of 4 ml/min.

Isoelectric Precipitation.

40 mg of zinc chloride was added to a pooled fraction of insulin containing 141 mg of insulin (the pools obtained from the above chromatographic process). The pH was raised to 6.0 with sodium hydroxide in order to precipitate insulin from the pool as zinc insulin. The precipitate was kept at 8° C. for 12 hours followed by centrifugation and then dried to isolate zinc insulin.

FIG. 1: Schematic presentation of the secretion and processing of the insulin pre-pro polypeptide possessing the KEX site in the signal sequence region

FIG. 2: Schematic presentation of the secretion and processing of insulin pre-pro polypeptide not having the KEX site in the signal sequence region. In this example, there is a single basic amino acid residue (Arg) just adjacent to the insulin polypeptide region

FIG. 3: Describes the expression vector (the “Vector Map”) used for the expression and secretion of heterlogous proteins using the present invention. MOX-promoter refers to the alcohol inducible promoter methanol oxidase promoter, MOX-T refers to the methanol oxidase terminator. Amp refers to the amplicillin resistance conferring gene and URA3 is the yeast auxotropic selection marker. The vector map includes the locations of the various restriction endonuclease sites of the vector.

We claim:

1) A DNA construct having a formula
pY-SP-B(1-29)-A(1-21),
where A) pY is any promoter in yeast, B) SP encodes a signal peptide region that enables the secretion of polypeptides expressed in yeasts, and is derived from either Schwanniomyces occidentalis glucoamylase signal peptide sequence or from Carcinus maenas crustacean hyperglycemic harmone signal peptide sequence, and lies to the N-terminus of the insulin peptide region B(1-29)-A(1-21) and C) B(1-29)-A(1-21) encodes, upon expression, the insulin peptide region in which B(1-29) is the B chain of insulin from amino acid 1 to amino acid 29, A(1-21) is the A chain of insulin from amino acid 1 to amino acid 21, and that the amino acid 29 of the B chain directly connects, by means of a peptide bond, the amino acid 1 of the A chain and the expression of SP-B(1-29)-A(1-21) region is under the control of the promoter—pY.

2) A DNA construct according to claim 1 where the SP is derived from Schwanniomyces occidentalis glucoamylase signal peptide sequence.

3) A DNA construct according to claim 1 where the SP is derived from Carcinus maenas crustacean hyperglycemic harmone signal peptide sequence.

4) A DNA construct according to claim 2 in which the SP carries a kex protease cleavage site.

5) A DNA construct according to claim 3 in which the SP carries a kex protease cleavage site.

6) A DNA construct according to claim 2 in which the SP does not carry any kex protease cleavage site.

7) A DNA construct according to claim 3 in which the SP does not carry any kex protease cleavage site.

8) A DNA construct according to claim 6 in which the SP has a single methionine residue placed such that it is just adjacent and N-terminus to the polypeptide encoded by the insulin peptide region B(1-29)-A(1-21).

9) A DNA construct according to claim 7 in which the SP has a single methionine residue placed such that it is just adjacent and N-terminus to the polypeptide encoded by the insulin peptide region B(1-29)-A(1-21).

10) A DNA construct according to claim 6 in which the SP has either a single Arginine or a single Lysine residue placed such that it is just adjacent and N-terminus to the polypeptide encoded by the insulin peptide region B(1-29)-A(1-21).

11) A DNA construct according to claim 7 in which the SP has either a single Arginine or a single Lysine residue placed such that it is just adjacent and N-terminus to the polypeptide encoded by the insulin peptide region B(1-29)-A(1-21).

12) A polypeptide SP-B(1-29)-A(1-21) B(1-29)-A(1-21), where SP is a signal peptide region that enables the secretion of polypeptides expressed in yeasts and is derived from either Schwanniomyces occidentalis glucoamylase signal peptide sequence or from Carcinus maenas crustacean hyperglycemic harmone signal peptide sequence, and lies to the N-terminus of the insulin peptide region B(1-29)-A(1-21), and further where B(1-29) is the B chain of insulin from amino acid 1 to amino acid 29, A(1-21) is the A chain of insulin from amino acid 1 to amino acid 21, and the amino acid 29 of the B chain directly connects, by means of a peptide bond, the amino acid 1 of the A chain.

13) A polypeptide according to claim 12 where the SP is derived from Schwanniomyces occidentalis glucoamylase signal peptide sequence.

14) A polypeptide according to claim 12 where the SP is derived from Carcinus maenas crustacean hyperglycemic harmone signal peptide sequence.

15) A polypeptide according to claim 13 in which the SP carries a kex protease cleavage site.

16) A polypeptide according to claim 14 in which the SP carries a kex protease cleavage site.

17) A polypeptide according to claim 13 in which the SP does not carry any kex protease cleavage site.

18) A polypeptide according to claim 14 in which the SP does not carry any kex protease cleavage site.

19) A polypeptide according to claim 17 in which the SP has a single methionine residue placed such that it is just adjacent and N-terminus to the polypeptide encoded by the insulin peptide region B(1-29)-A(1-21).

20) A polypeptide according to claim 18 in which the SP has a single methionine residue placed such that it is just adjacent and N-terminus to the polypeptide encoded by the insulin peptide region B(1-29)-A(1-21).

21) A polypeptide according to claim 17 in which the SP has either a single Arginine or a single Lysine residue placed such that it is just adjacent and N-terminus to the polypeptide encoded by the insulin peptide region B(1-29)-A(1-21).

22) A polypeptide according to claim 18 in which the SP has either a single Arginine or a single Lysine residue placed such that it is just adjacent and N-terminus to the polypeptide encoded by the insulin peptide region B(1-29)-A(1-21).

23) A DNA construct according to claim 1 in which the promoter, pY, is of yeast origin.

24) A DNA construct according to claim 23 in which the promoter, pY, is either the methanol oxidase promoter (MOX-P) or Formaldehyde dehydrogenase promoter (FMDH-P) or Formate dehydrogenase promoter (FMD-P) or Dihydroxyacetone synthase promoter (DHAS-P).

25) A process for the expression of insulin in yeasts which consists of transforming the said yeast with a plasmid that carries the DNA construct of claim 1, culturing the said transformed yeasts in an appropriate culture and isolating the insulin containing polypeptide from the culture medium.

26) A process according to claim 25 where the yeast is selected from genera Hansenula, Saccharomyces, Pichia, Kluyveromyces.

27) A process according to claim 26 where the yeast is Hansenula polymorpha.

28) A DNA construct of claim 1 in which B(1-29) is the B chain of human insulin from amino acid 1 to amino acid 29, A(1-21) is the A chain of human insulin from amino acid 1 to amino acid 21.

29) Process for the isolation, purification and conversion to native insulin, of the polypeptides of claims 15 consisting of the following steps:
a) Clarification of the culture supernatants containing the above polypeptides.
b) Subjecting the clarified culture supernatants to cation exchange chromatography.
c) Isoelectric precipitation of the cation exchange chromatography derived polypeptides.
d) Transpeptidation reaction in which the polypeptide precipitates were converted to insulin-t-butyl ester-t-butyl ether.
e) Purification of the insulin-t-butyl ester-t-butyl ether, by reverse phase chromatography.
f) Hydrolysis of the insulin-t-butyl ester-t-butyl ether to native insulin.
g) Purification of insulin wherein the insulin obtained from the hydrolysis reaction was purified on a reverse phase HPLC column.
h) Isoelectric precipitation of the purified insulin.

30) A process according to claim 29 where any two steps are performed in sequence.

31) Process for the isolation, purification and conversion to native insulin, of the polypeptides of claim 16 consisting of the following steps:
a) Clarification of the culture supernatants containing the above polypeptides.
b) Subjecting the clarified culture supernatants to cation exchange chromatography.
c) Isoelectric precipitation of the cation exchange chromatography derived polypeptides.
d) Transpeptidation reaction in which the polypeptide precipitates were converted to insulin-t-butyl ester-t-butyl ether.
e) Purification of the insulin-t-butyl ester-t-butyl ether, by reverse phase chromatography.
f) Hydrolysis of the insulin-t-butyl ester-t-butyl ether to native insulin.
g) Purification of insulin wherein the insulin obtained from the hydrolysis reaction was purified on a reverse phase HPLC column.
h) Isoelectric precipitation of the purified insulin.

32) A process according to claim 31 where any two steps are performed in sequence.

33) Process for the isolation, purification and conversion to native insulin, of the polypeptides of claim 21 consisting of the following steps:
a) Clarification of the culture supernatants containing the above polypeptides.
b) Subjecting the clarified culture supernatants to cation exchange chromatography.
c) Isoelectric precipitation of the cation exchange chromatography derived polypeptides.
d) Transpeptidation reaction in which the polypeptide precipitates were converted to insulin-t-butyl ester-t-butyl ether.
e) Purification of the insulin-t-butyl ester-t-butyl ether, by reverse phase chromatography.
f) Hydrolysis of the insulin-t-butyl ester-t-butyl ether to native insulin.
g) Purification of insulin wherein the insulin obtained from the hydrolysis reaction was purified on a reverse phase HPLC column.
h) Isoelectric precipitation of the purified insulin.

34) A process according to claim 33 where any two steps are performed in sequence.

35) Process for the isolation, purification and conversion to native insulin, of the polypeptides of claim 22 consisting of the following steps:
a) Clarification of the culture supernatents containing the above secreted polypeptides.
b) Subjecting the clarified culture supernatents to cation exchange chromatography.
c) Isoelectric precipitation of the cation exchange chromatography derived polypeptides.
d) Transpeptidation reaction in which the polypeptide precipitates were converted to insulin-t-butyl ester-t-butyl ether.
e) Purification of the insulin-t-butyl ester-t-butyl ether, by reverse phase chromatography.
f) Hydrolysis of the insulin-t-butyl ester-t-butyl ether to native insulin.
g) Purification of insulin wherein the insulin obtained from the hydrolysis reaction was purified on a reverse phase HPLC column.
h) Isoelectric precipitation of the purified insulin.

36) A process according to claim 35 where any two steps are performed in sequence.
